(19) United States
(12) Patent Application Publication
(10) Pub. No.: US 2016/0022775 A1
(43) Pub. Date: Jan. 28, 2016

(54) TREATING LYSOSOMAL STORAGE DISEASE

(71) Applicant: UNIVERSITY OF ROCHESTER, Rochester, NY (US)

(72) Inventors: Mark Noble, Rochester, NY (US); Christopher Folts, Rochester, NY (US); Nicole Scotts, West Henrietta, NY (US)

(73) Assignee: UNIVERSITY OF ROCHESTER, Rochester, NY (US)

(21) Appl. No.: 14/777,100

(22) PCT Filed: Mar. 14, 2014
(86) PCT No.: PCT/US2014/028209

§ 371 Date: Sep. 15, 2015

Related U.S. Application Data
(60) Provisional application No. 61/792,854, filed on Mar. 15, 2013.

(51) Int. Cl. A61K 38/18; G01N 33/50; A61K 31/352; A61K 31/085; A61K 31/05

(52) U.S. Cl. CPC A61K 38/1866; A61K 31/085; A61K 31/05; A61K 31/352; A61K 38/18; G01N 33/5058

(57) ABSTRACT

Provided herein are compositions, kits and methods related to the treatment of one or more lysosomal storage diseases (LSDs) in a subject and methods of identifying agents that are useful for the treatment of one or more LSDs. The compositions, kits, and methods are based on the novel discovery that agents effective for treating LSD must have two characteristics: they must inhibit cell death and reduce suppression of cell division caused by toxic substances that accumulate in LSD. Exemplary agents include polypeptides (e.g., IGF-1, VEGF) and small molecules (e.g., chlorotrianisene, clofoctal, colforsin, and tulobuterol).

This application claims the benefit of U.S. Application No. 61/792,854, filed on Mar. 15, 2013, which is hereby incorporated in its entirety by this reference.

### STATEMENT REGARDING FEDERALLY FUNDED RESEARCH

This invention was made with government support under grant number 1F31NS078911 awarded by the National Institutes of Health. The government has certain rights in the invention.

### BACKGROUND

Lysosomal storage disorders represent some of the most devastating of genetic diseases, and the need to develop therapies for these disorders remains largely unmet. For many of these diseases, damage to the central nervous system (CNS) is of central importance, but the mechanisms underlying such damage are largely unknown. Moreover, because each of the lysosomal storage disorders is rare (less than about 1:100,000 individuals is affected), with the total of all lysosomal storage diseases affecting about 1 in every 5,000 to 10,000 individuals, research on these problems has been limited. Nonetheless, the severity of the effects and the limited treatment options present a serious medical burden.

### SUMMARY

Provided herein are compositions, kits and methods related to the treatment of one or more lysosomal storage diseases (LSDs) in a subject and methods of identifying agents that are useful for the treatment of one or more LSDs. The compositions, kits, and methods are based on the novel discovery that agents effective for treating LSD must have two characteristics: they must inhibit cell death and reduce suppression of cell division caused by toxic substances that accumulate in LSD. A novel assay system was utilized wherein the characteristics are demonstrated in primary central nervous system cells (e.g., precursor cells like glial precursor cells).

A method of treating LSD comprises administering to the subject an effective amount of a first agent that inhibits cell death and reduces suppression of cell division of primary central nervous system precursor cells in the presence of one or more toxic substances, wherein the toxic substances are known to accumulate in cells affected by lysosomal storage disease. Optionally, the first agent inhibits cell death and reduces suppression of cell division of primary central nervous system precursor cells in the presence of at least two toxic substances. The method can further comprise administering to the subject a second agent that inhibits cell death and reduces suppression of cell division of primary central nervous system precursor cells in the presence of one or more toxic substances.

The method of treating a lysosomal storage disease in a subject can comprise selecting one or more agents that inhibit cell death and reduces suppression of cell division of primary central nervous system precursor cells in the presence of one or more toxic substances, wherein the toxic substances are known to accumulate in cells affected by lysosomal storage disease, and administering an effective amount of the one or more agents to the subject.

Also provided is a method of identifying an agent for treating LSD, comprising detecting a level of cell survival of a primary central nervous system cell in the presence of one or more toxic substances, wherein the one or more toxic substances are known to accumulate in cells affected by lysosomal storage disease; detecting a level of cell division of a primary central nervous system cell in the presence of the one or more toxic substances. An agent that promotes the level of cell survival and promotes the level of cell division of the primary central nervous system cell in the presence of the one or more toxic substances as compared to the level of cell survival and cell division in the absence of the agent is an agent useful for treating lysosomal storage disease. An agent identified according to this method can be used in the treatment methods and composition and kits disclosed herein. The steps of detecting survival and division of the primary central nervous system cell are optional performed in vitrousing a central nervous system precursor cell.

### DESCRIPTION OF DRAWINGS

FIG. 1 shows a graph of the results in assays for survival (left), proliferation (center) and clonal expansion (right) in precursor cells that can give rise to oligodendrocytes (oligodendrocyte/type-2 astrocyte progenitor or oligodendrocyte precursor cells (O-2A/OPCs) (“OPC”) in the presence of psychosine. Micromolar concentrations of psychosine (PC) induce cell death of OPCs and, at lower concentrations, suppression of progenitor cell division. Purified OPCs were exposed to PC at various concentrations for 1, 3 or 5 days and cell survival was determined by calcein AM labeling and analysis by CELIGO® (Chelmsford, Mass.) analysis. As shown, by day 3 even 3.3 μM PC caused marked cell death. Division was analyzed by clonal analysis, and exposure to 1 μM PC caused a marked reduction in clonal size for cells grown in the presence of PDGF for 6 days.

FIG. 2 shows a photomicrograph (left) and a graph (right) of OPCs in culture exposed to PDGF (to promote migration) in the presence of PC. Exposure of oligodendrocyte progenitor cells to 1 μM psychosine is enough to greatly reduce cell migration.

FIG. 3 is a graph showing results from a cell survival assay using a 1040 compound library to determine which agents rescue cell survival in the presence of psychosine. Forty compounds that caused significant rescue of survival from the lethal effects of psychosine exposure were identified from the library, which included drugs that are approved or in the clinic.

FIG. 4 shows graphs of proliferation (top) and cell survival (bottom) using multiple protein (polypeptides) to assess rescue of OPCs exposed to psychosine. The results of screening multiple proteins found few that rescue cell division and fewer that impact survival. The proteins examined were platelet-derived growth factor (PDGF), fibroblast growth factor-2 (FGF-2), epidermal growth factor (EGF), hepatocyte growth factor (HGF), neurotrophin-3, glial-derived neurotrophic factor (GDNF), glial growth factor (neuregulin), vascular endothelial growth factor (VEGF), insulin-like growth factor-1 (IGF-1) and insulin-like growth factor-2 (IGF-2). Only IGF-1 and VEGF had the ability to protect both cell division and cell survival.

FIG. 5 shows graphs of self-renewing division (left) and cell survival (right) of primary OPCs exposed to psychosine and IGF. IGF-1 rescued both division and survival of primary OPCs. These data show that beneficial effects on survival are seen at dosages even as low as 10 ng/ml. Rescue of cell division is also achieved at physiologically relevant dosages of IGF-1, something that has not been previously observed.

FIG. 6 shows a graph of cell survival at various concentration of psychosine, glucopsychosine, or lysosulfatide. Glucopsychosine and lysosulfatide are toxic for primary oligodendrocyte progenitor cells (OPCs) and other primary cells of the central nervous system at exposure levels in the low micromolar range. This is the same range at which toxicity of psychosine is seen.

FIG. 7 shows a bar graph of cell survival of OPCs exposed to either glucopsychosine (left) and lysosulfatide (right) in the presence of a bioactive protein. Data are shown for PDGF, bFGF, IGF-1, HGF or NT-3. The bioactive proteins were screened for their ability to (1) protect against psychosine-mediated suppression of cell division and cell survival and (2) protect against the toxicity of glucopsychosine and lysosulfatide. Of these proteins, only IGF-1 protects against psychosine mediated suppression of cell division and cell survival and protects against the toxicity of glucopsychosine and lysosulfatide.

FIG. 8 shows a bar graph of locomotor (left) and OPC cell division (right) from twi−/− mice treated with saline or IGF, IGF-I rescues twi−/− mice locomotor behavior and OPC division. Quantification of the velocity of movement (cm/sec) of IGF-I treated mice shows that IGF-I restores movement to normal levels (*=p, 0.05; n.s.=not significant). Analysis of OPC division, with outcomes normalized to division in IGF-I treated mice, shows that there is ˜45% more dividing OPCs in these mice than in saline-treated controls.

### DETAILED DESCRIPTION

Lysosomal storage disorders are caused by mutations involved in processing a variety of lipids and glycosaminoglycans. These mutations each lead to accumulation of a specific metabolite, or metabolites, each of which is specific to the particular disorder. These diseases can be functionally grouped in several different ways. LSDs can be characterized according to the tendency of fibroblasts obtained from the affected patients to accumulate BODIPY-LacCer in lysosomes (Chen et al., The Lancet 354: 9182: 901-905 (1999). According to this particular outcome measure, Krabbe disease, Gaucher's disease and Farber's disease accumulate BODIPY-LacCer and are considered distinct from multiple other lysosomal storage disorders (including metachromatic leukodystrophy). Alternatively, LSDs are characterized by the primary type of material that accumulates in the specific disorder. Thus, lipid storage disorders include sphingolipidoses (including Gaucher's and Niemann-Pick diseases) gangliosidosis (including Tay-Sachs disease), and leukodystrophies; mucopolysaccharidoses (including Hunter syndrome and Hurler disease); glycoprotein storage disorders; mucolipidoses; and glycogen storage disease type II (Pompe disease).

The history of research on lysosomal storage disease therapies has been focused on the characteristics of each individual disease, as typified by the attempts to develop enzyme replacement therapy for each disease in disease-specific animal models. This highly specific approach to therapy is exemplified by the treatment of Gaucher's disease with delivery of the enzyme mutated in this disease, an approach that would not be utilized for treatment of any other lysosomal storage disorder. This is also the case for experimental treatments involving gene therapy to modify cells to deliver the enzyme that is defective for that specific disease.

Another example of the specificity accorded to each disease is that of using substrate reduction therapy to reduce the levels of the substrate required to produce the putatively toxic substance accumulated in each disease.

Thus, the analysis of lysosomal storage disorders can be seen to be strongly biased against the hypothesis that there might exist general therapeutic approaches in which single therapeutic agents might have relevance to multiple different diseases. Even the sole general approach that has been employed in some clinical cases and in experimental treatment strategies, that of bone marrow transplant, is predicated on the theoretical ability of cells derived from a normal transplant to secrete and thus deliver the specific missing mutant enzyme relevant to that individual disease. Thus, even though a single therapeutic approach (bone marrow transplantation) is utilized in these cases, it is nonetheless used with the intent of supplying replacement enzyme relevant to the specific mutation that characterizes each disease.

Krabbe disease (also called globoid cell leukodystrophy) is an autosomal recessive disorder caused by a deficiency of galactosylceramidase (GCase), an enzyme required for metabolism of specific galactolipids. Krabbe disease is characterized by inflammation, astrogliosis, death of oligodendrocytes and progressive loss of myelin, with neuronal pathologies in both the CNS and PNS, and with death of children with early infantile onset within the first years of life. In murine models of this disease, axonal pathology begins within 7 days after birth (p7), inflammation begins at P15, and oligodendrocyte death, myelin loss and neuronal death begins at P30, with death at about P25-45 (or P58 for a newer mouse model that retains a reduced level of GCase activity). Current treatments are limited to bone marrow transplantation (BMT) to introduce cells (mostly macrophages) into the CNS capable of producing GCase, and are only partially efficacious. Gene therapy in murine models, when combined with BMT, markedly reduces tissue damage and increases survival, although even these approaches do not prevent axonal pathologies. In mouse models, treatment with anti-inflammatory agents also can prolong survival and reduce severity of at least some of the pathological changes that occur in these animals.

Molecular mechanisms underlying tissue damage in Krabbe disease are far from clear, but there is widespread agreement that psychosine (PC), a galactolipid that accumulates in this disease, can itself cause oligodendrocyte and neuron death, collapse of neuronal processes and inflammation. PC has multiple effects, and can accumulate in lipid rafts and inhibit raft-mediated endocytosis, inhibit protein kinase C activity (possibly through its effects on lipid rafts), activate secretory phospholipase A2, stimulate activity of jun kinase and AP-1 via upregulation of reactive oxidative species (ROS) leading to oligodendrocyte death, inhibit peroxisomal and mitochondrial function, reduce activity of AMP-activated protein kinase (AMPK), alter lipid biosynthesis and induce expression of proinflammatory cytokines and iNOS in primary astrocytes. Previous studies (See Zaka et al., Mol. Cell Neurosci. 30 (3): 398-407 (2005)) suggest that the precursor cells that give rise to oligodendrocytes (oligodendrocyte/type-2 astrocyte progenitor or oligodendrocyte precursor cells (O-2A/OPCs)) are vulnerable to psychosine (PC) at concentrations of 50 μM, a concentration well above that which affects oligodendrocytes and neurons.

Gaucher's disease is caused by defects in lysosomal glucocerebrosidase (also known as beta-glucosidase), leading to accumulation of glucopsychosine. There are three clinical sub-types with relatively little brain involvement (Type I), or severe neurological damage (Types II and III). Type II is defined as having acute infantile onset, with death usually occurring by age 2. Types I and III disease can begin early in life or in adulthood. Prognosis for Type II disease is dismal and individuals with Type III disease rarely survive beyond 30 years of age. Enzyme replacement therapy is useful in Types I and III disease, but at a cost of $200,000/year. Miglustat, a synthetic analogue of D-glucose that inhibits glucosylceramide synthase, has also been approved for use in Type I Gaucher's patients. These are therapies that are very specific for treatment of Gaucher's disease.

Metachromatic leukodystrophy is caused by mutations in arylsulfatase A that lead to accumulation of sulfatide species (primarily lysosulfatide). MLD can present with infantile, early or adult onset. Children with infantile onset rarely survive beyond 5 years of age, and those with juvenile onset rarely survive more than 10-15 years after onset. Neurological complications are severe, and are particularly seen as damage to myelinated tracts. There is no treatment, although bone marrow transplantation is under investigation.

In contrast to current therapies, provided herein are methods of treating a lysosomal storage disorder or disease (LSD) using agents useful in a variety of LSDs and methods of screening for such agents. These agents and methods turn on the identification of agents that both inhibit cell death and reduce suppression of cell division of primary central nervous system precursor cells in the presence of one or more toxic substances, preferably associated with more than one LSD.

### Methods of Treatment

Thus provided herein is a method of treating an LSD in a subject comprising administering to the subject an effective amount of a first agent that inhibits cell death and reduces suppression of cell division of primary central nervous system precursor cells in the presence of one or more toxic substances, wherein the toxic substances are known to accumulate in cells affected by the lysosomal storage disease. As a preliminary step, the method can involve selecting a subject with or at risk of developing an LSD. Subjects can be selected based on the onset of symptoms, but, more preferably based on a family history and/or a genetic test, so that treatment can be initiated as early as possible and before substantial cell damage or CNS damage has occurred.

Optionally, the first agent used in the method is selected from the group consisting of a polypeptide (e.g., IGF-1 or VEGF) or a small molecule (e.g., chlorotrianisene, clofoctol, tulobuterol or colforsin), which are shown herein to inhibit cell death and reduce suppression of cell division of primary central nervous system precursor cells in the presence of one or more toxic substances associated with LSD. Other agents that have the capacity to both inhibit cell death and reduce suppression of cell division of primary central nervous system precursor cells in the presence of one or more toxic substances, which include, without limitation, glucopsychosine, lysosulfatide, and psychosine), could be used in the methods taught herein. The ability of the agent to inhibit cell death and reduce suppression of cell division of primary central nervous system precursor cells optionally occurs in the presence of more than one toxic substance, wherein the multiple toxic substances are associated with different LSDs. For example, the agent optionally inhibits cell death and reduces suppression of cell division of primary central nervous system precursor cells in the presence of either glucopsychosine and lysosulfatide, glucopsychosine and psychosine, and/or lysosulfatide and psychosine. Thus, the first agent used for treatment inhibits cell death and reduces suppression of cell division of primary central nervous system precursor cells in the presence of at least two (e.g., 2, 3, 4, 5, 6, 7, 8, 9, 10, or more) toxic substances, wherein the two toxic compounds are associated with different LSDs.

Optionally, the method of treatment further comprises administering to the subject a second agent that inhibits cell death and reduces suppression of cell division of primary central nervous system precursor cells in the presence of one or more toxic substances. The second agent optionally has the same characteristics as the first agent but is a different agent. Thus, the first and second agents can be selected from any combination of IGF-1, VEGF, chlorotrianisene, clofoctal, tulobuterol and colforsin. Optionally, the method of treatment further comprises administering to the subject a third, fourth, and/or fifth agent that inhibits cell death and reduces suppression of cell division of primary central nervous system precursor cells in the presence of one or more toxic substances. The various additional agents may be effective against different toxic agents from different LSDs, thus offering a broad cocktail of agents useful in a variety of LSDs.

Thus provided herein is a method of treating a lysosomal storage disease in a subject that includes selecting one or more agents that inhibit cell death and reduce suppression of cell division of primary central nervous system precursor cells in the presence of one or more toxic substances, wherein the toxic substances are known to accumulate in cells affected by lysosomal storage disease, and administering an effective amount of the one or more agents to the subject.

The LSD to be treated by the methods herein is optionally a sphingolipid storage disorder (e.g., selected from the group including, but not limited to gloiboid-cell leukodystrophy (Krabbe Disease), metachromatic leukodystrophy, and Gaucher's Disease) or other varieties thereof. However, a wide variety of LSDs could be treated with the agents or combinations of agents taught herein. LSDs include, but are not limited to, Activator Deficiency/GM2 Gangliosidosis, Alpha-mannosidosis, Aspartylglucosaminuria, Cholesteryl ester storage disease, Chronic Hexosaminidase A Deficiency, Cystinosis, Danon disease, Fabry disease, Farber disease, Fucosidosis, Galactosialidosis, Gaucher Disease (Type I, II and III), GM1 gangliosidosis (infantile, late infantile/juvenile and adult/chronic), I-Cell disease/Mucolipidosis II, Infantile Free Sialic Acid Storage Disease/ISSD, Juvenile Hexosaminidase A Deficiency, Krabbe disease (infantile onset late onset), Lysosomal acid lipase deficiency, Metachromatic Leukodystrophy, Mucopolysaccharidoses disorders (for example, Pseudo-Hurler polydystrophy/Mucolipidosis IIIA, MPSI Hurler Syndrome, MPSI Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type A/MPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC, Mucolipidosis type IV), Metachromatic leukodystrophy, Multiple sulfatase deficiency, Niemann-Pick Disease

(Type A, B and C), Neuronal Ceroid Lipofuscinoses (for example, CLN6 disease (Atypical Late Infantile, Late Onset variant, Early Juvenile), Batten-Spielmeyer-Vogt/Juvenile NCL/CLN3 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis, Pompe disease/Glycogen storage disease type II, Pycnodysostosis, Sandhoff disease/Adult Onset/GM2 Gangliosidosis, Sandhoff disease/GM2 gangliosidosis-Infantile, Sandhoff disease/GM2 gangliosidosis (juvenile), Schindler disease, Salla disease/Sialic Acid Storage Disease, Tay-Sachs/GM2 gangliosidosis and Wolman disease. A mutation in the glucocerobrosidase gene, associated with Gaucher's Disease is also associated with Parkinson's disease.

The cells of a subject with a lysosomal storage disorder often have a pH that is above normal. In the methods provided herein, the agent optionally restores the pH of the lysosomes to or toward normal pH, which is below about 5.0. As utilized throughout, normal lysosomal pH is from about 4.5 to about 5.0. In the methods set forth herein, the cells of the subject, for example, central nervous system cells (CNS), can have above normal pH as a result of exposure to a toxic substance associated with a lysosomal storage disorder. These substances include lipids, and more specifically include, without limitation, psychosine, glucopsychosine and lysosulfatide. Restoration of normal lysosomal pH can be associated with a reduction or with prevention of cell death and/or a reduction or prevention of a suppression of cell division. Thus, in the methods provided herein, the agent used to treat LSD optionally restores lysosomal pH to normal levels.

By way of example, the one or more agents used in the treatment of LSD in a subject can be VEGF and the LSD is metachromatic leukodystrophy or Gaucher's Disease but not gloiboid-cell leukodystrophy (Krabbe Disease). However, the one or more agents including VEGF could be effective against both metachromatic leukodystrophy and Gaucher's Disease and, optionally against Krabbe as well.

Administration of the agents and compositions described herein or pharmaceutically acceptable salts thereof can be carried out using therapeutically effective amounts of the compounds and compositions described herein or pharmaceutically acceptable salts thereof as described herein for periods of time effective to treat a disorder. An effective amount is selected so as to provide sufficient therapeutic effect with minimal to no unwanted side-effects. The effective amount of the compounds and compositions described herein or pharmaceutically acceptable salts thereof as described herein may be determined by one of ordinary skill in the art and includes exemplary dosage amounts for a mammal of from about 0.01 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

In the case of IGF-1, the dosage is optionally about 0.01 to about 0.40 mg/kg daily (or any amount in between), but can be administered in multiple doses (e.g., twice daily).

Optionally the dosage is about 0.01-0.12 mg/kg at least once or twice daily or can be about 0.08-0.24 mg/kg daily. In the case of VEGF, the dosage is optionally about 0.3 to about 6.0 mg/kg (or any amount in between) daily, but can be administered in multiple doses (e.g., twice daily). In the case of chlorotrianisene, the dosage is optionally about 0.05 to about 4 mg/kg (or any amount in between) daily, but can be administered in multiple doses. In the case of clofoctal, the dosage is optionally about 1 to about 20 mg/kg (or any amount in between) daily, but can be administered in multiple doses. In the case of colforsin, the dosage is optionally about 0.1 to about 300 mg/kg (or any amount in between) daily, but can be administered in multiple doses.

As used herein, IGF-1, VEGF, chlorotrianisene, clofoctal, tulobuterol and colforsin are exemplary only. Other agents meeting the required characteristics, as well as modified, active versions of IGF-1 and VEGF or derivatives of chlorotrianisene, clofoctal, tulobuterol and colforsin are contemplated for use herein. For example, modified, active forms of IGF-1 and VEGF can include truncation, mutations, substitutions, and deletions. By way of example, conservative amino acid substitutions can be made in one or more of the amino acid residence of the polypeptide to generate an active version thereof Derivatives of the small molecules can also be designed by one of skill in the art by changing, removing, or adding substituent groups. It is within the skill in the art to test the modified version and derivative for the desired activities.

One or more of the agents described herein or identified with the method described herein or derivatives thereof can be provided in a pharmaceutical composition, in a kit with one or more of the agents and a means of administering the agent or agents to the subject (e.g., a syringe, a patch, or IV system). The pharmaceutical compositions are administered in a number of ways depending on whether local or systemic treatment is desired, and on the area to be treated. The pharmaceutical compositions are administered via any of several routes of administration, including topically, orally, parenterally, intravenously, intra-articularly, intraperitoneally, intramuscularly, subcutaneously, intradermally, intracavity (e.g., rectal, intravesical, lumen of vesical organs), transdermally, intrahepatically, intracranially, nebulization/inhalation, or by installation via bronchoscopy. Intradermal administration includes administration at a site that is afferent to the site of lymphatic transport dysfunction. Optionally, the pharmaceutical composition is administered by oral inhalation, nasal inhalation, intranasal mucosal administration, or suppository. The pharmaceutical composition can also be injected or infused, for example, at a site of inflammation, such as, for example, an inflamed joint. Administration of the pharmaceutical compositions by inhalant can be through the nose or mouth via delivery by spraying or droplet mechanism, for example, in the form of an aerosol. The agents described herein are optionally provided by injection (e.g., by subcutaneous injection) or by a pump for continuous or nearly continuous administration.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, N.J.), polyethylene glycol (PEG), and PLURONICS™ (BASF; Florham Park, N.J.).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

The method disclosed herein optionally can be combined with one or more treatments, such that the subject is further treated with one or more treatments selected from the group consisting of a bone marrow transplant, enzyme replacement, and gene therapy. Thus, by way of example, the subject can be treated with one or more agents that inhibit cell death and reduce suppression of cell division of primary central nervous system precursor cells in the presence of one or more toxic substances, wherein the toxic substances are known to accumulate in cells affected by lysosomal storage disease, and could also be treated with one or more additional therapeutic measures.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous or sequential administration of two or more agents or compositions.

### Methods of Identifying Agents Useful in Treating One or More Lysosomal Storage Diseases

Provided herein is a method of screening for an agent for treating lysosomal storage disease (LSD) comprising detecting a level of cell survival of a primary central nervous system cell in the presence of one or more toxic substances, wherein the one or more toxic substances are known to accumulate in cells affected by a lysosomal storage disease; and detecting a level of cell division of a primary central nervous system cell in the presence of the one or more toxic substances, e.g., substances known to accumulate in LSD. An agent that increases the level of cell survival and increases the level of cell division of the primary central nervous system cell in the presence of the one or more toxic substances as compared to the level of cell survival and cell division in the absence of the agent is an agent that treats lysosomal storage disease. The steps of detecting survival and division of the primary central nervous system cell can be performed in vitro and the primary central nervous system cell is optionally a precursor cell, such as a glial precursor cell (e.g., a glial precursor cell may be capable of giving rise to an oligodendrocyte and, more specifically, an oligodendroctye-type-2 astrocyte (O-2A) precursor cell, which, unlike cell lines studied to date, shows physiologic sensitivity to toxic substances that accumulate in LSDs; but may also include glial-restricted precursor cells, neuroepithelial stem cells or other CNS precursor cells.

The detection of cell division and the detection of cell survival can be performed using any number of methods known in the art and described in the Examples. For example, to analyze cell survival, the general strategies applied are to expose cells for 1, 3 or 5 days to a two order-of-magnitude dose response of agents that accumulate in lysosomal storage disorders. Cells used are primary dividing cells of the central nervous system and may include oligodendrocyte progenitor cells from postnatal animals, embryonic glial-restricted precursor cells, neuron-restricted precursor cells, neuroepithelial stem cells (NSCs) or Schwann cells from rodent or human nervous system. Cells are plated at varying densities onto appropriate tissue culture surfaces (as defined in the art and well known to practitioners in the art) and cell division may be analyzed by labeling with the Ki67 antibody, incorporation of uridine or thymidine derivatives, increases in cell number or any other methods known to the art. Cell death is analyzed similarly except that assays may be extended to included neurons from the central or peripheral nervous system. Cell death may be determined by using calcein AM and propidium iodide (PI) labeling of live cultures to identify live and dead cells, TUNEL staining, DAPI staining, visual inspection, analysis of metabolic activity (e.g., with MTT or Alamar Blue), activation of caspase-3 or other caspases or any other methods widely known to those skilled in the art.

The one or more toxic substances are described above and can include one or more toxic lipids. Multiple toxic substances are preferably associated with different LSDs and can be tested together or separately in the assay steps. The period of time for contact between the toxic agent and the cells can be determined by one skilled in the art using the teachings herein.

As used throughout, the agent to be screened or the selected agent can be a small molecule, for example, a compound, a drug, or a chemical that reduces or prevents cell death and reduces or prevents suppression of cell division. The agent can also be a polypeptide, or an active fragment thereof. The polypeptide can be a purified or a recombinant polypeptide.

Suitable test compounds for use in the screening assays can be obtained from any suitable source, such as conventional compound libraries. The test compounds can also be obtained using any of the numerous approaches in combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The biological library approach includes peptide libraries, while the other four approaches include peptide, non-peptide oligomer, or small molecule libraries of compounds. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries and libraries of natural compounds in the form of bacterial, fungal, plant, and animal extracts are commercially available. In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

Examples of methods for the synthesis of molecular libraries can be found in the art. Libraries of compounds may be presented in solution or on beads, bacteria, spores, plasmids or phage.

A screening assay of the disclosure is particularly amenable to a high throughput format, thereby providing a means to screen, for example, a combinatorial library of small organic molecules, peptides, nucleic acid molecules, and the like.

As used herein, the term subject can be a vertebrate, more specifically a mammal (e.g., a human, horse, pig, rabbit, dog, sheep, goat, non-human primate, cow, cat, guinea pig or rodent), a bird, a reptile or an amphibian. The term does not denote a particular age or sex. Thus, adult and newborn subjects, whether male or female, are intended to be covered. As used herein, patient or subject may be used interchangeably and can refer to a subject with a disease or disorder. The term patient or subject includes human and veterinary subjects.

As used throughout primary central nervous system cells are cells derived directly from the central nervous system of a test subject, for example, and without limitation, a rat, mice, or other laboratory animal. By “primary” is meant that the cells are not cells from cell lines, such as immortalized cell lines. Primary central nervous system cells include cells capable of cell division such as precursor or progenitor cells. Primary central nervous system precursor cells include glial precursor cells, such as those that give rise to astrocytes or astrocytes and oligodendrocytes. One example of a glial precursor cell is the precursor cells that give rise to oligodendrocytes (called oligodendrocyte/type-2 astrocyte progenitor or oligodendrocyte precursor cells, and herein abbreviated as O-2A/OPCs or OPCs). Other examples of glial precursor cells are glial-restricted precursor cells, astrocyte-restricted precursor cells or neuroepithelial stem cells.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs (e.g., a reduction in muscle weakness) of the disease in a subject as compared to a control. As used herein, control refers to the untreated condition. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1

### In Vitro Studies

### Cellular and Molecular Analyses

Whether toxic lipids have similar effects on the function of oligodendrocyte precursor cells (OPCs, the direct ancestors of myelin-forming oligodendrocytes), due to the common features of myelin breakdown and failure to repair such damage in LSDs, was determined. These experiments all used standard techniques (see Mayer et al. PNAS, 91: 7496-7500 (1994); Ibarrola et al. Dev. Biol. 180: 1-121 (1996); Smith et al. PNAS 97: 10032-10037 (2000); Power et al. Dev. Biol. 245: 362-375 (2002); Dietrich et al. J. Biol. 5: e22 (2006); Li et al. PLoS Biol. 5: e35 (2007)), as adapted to enable rapid automated analysis of microscopy images captured using the CELIGO® imaging system (Brooks Automation, Chelmsford, Mass.). This system enables collection of data using brightfield and three-color fluorescence and provides similar information to be obtained at the single cell level in adherent cell cultures as yielded by flow cytometry. This approach enables quantitatively analysis of 80 different experimental conditions (in triplicate) per hour.

Studies on division and survival of OPCs exposed to 0.1, 0.5, 1, 2, 5 or 10 μM PC, GPS or LS were conducted for 1, 2, 3 or 5 days. These experiments followed standard procedures (see Mayer et al. PNAS, 91: 7496-7500 (1994); Dietrich et al. J. Biol. 5: e22 (2006); and Han et al. J. Biol. 7 (4):12 (2008)) except that cell death was quantified using calcein AM and propidium iodide (PI) labeling of live cultures was used to identify live and dead cells, respectively. While PI does not label cells that have lost their nuclei, it provides information on cells undergoing cell death and is also an internal control for calcein labeling (with which it is mutually exclusive). These dyes effectively overcome the problems with dyes for which cell numbers are inferred from metabolic activity (e.g., MTT, Alamar Blue), and provide quantitative information on cell numbers. Independent confirmation was obtained in all cases by visual examination of cultures. In these, and all other cases, all experiments were repeated at least three times, and all data points were collected in triplicate. All experiments were conducted in conditions of 5% O₂, to more closely mimic physiological oxygen concentrations.

The above experiments were supplemented by a focused molecular analysis of effects of putative toxic lipids on expression and activation status of proteins involved in cell division and cell survival.

### Toxicity of Disease-Relevant Lipids for CNS Progenitor Cells and Stem Cells

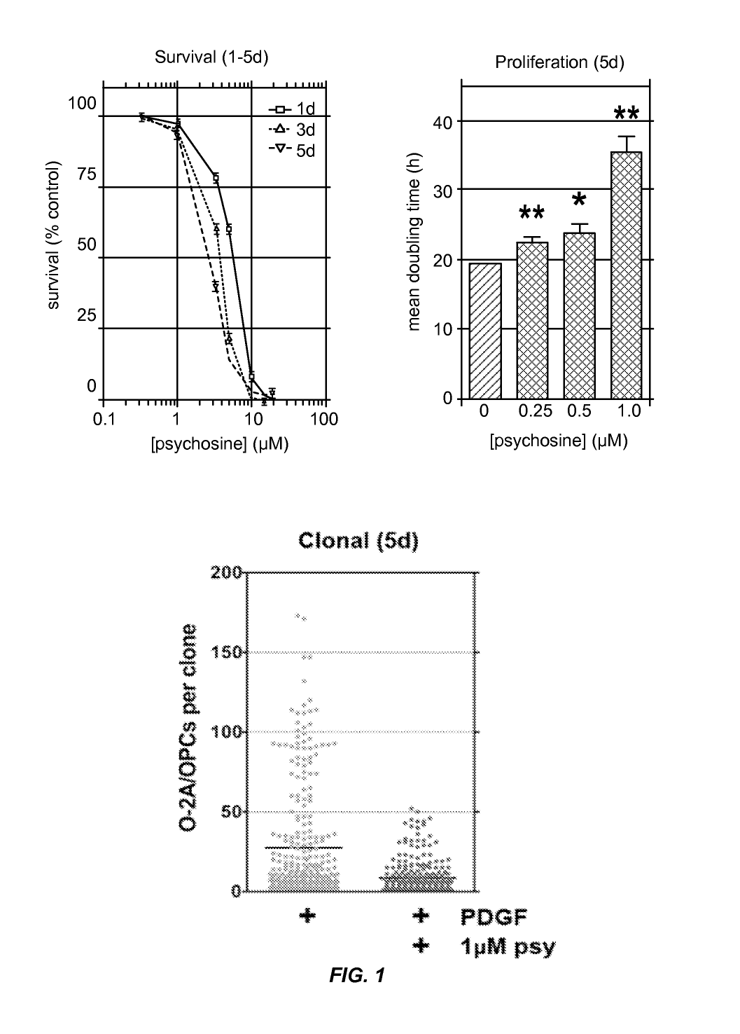

The sensitivity of primary OPCs to PC was determined. The sensitivity of the cells to glucopsychosine (GPC) and lysosulfatide (LS) was also examined. It was found that GPC and LS were toxic for OPCs at exposure levels as low as 3.304 and PC was toxic at levels as low as 5 μM (FIG. 1, data shown for PC). Thus, OPCs are even more sensitive to PC than oligodendrocytes, which are sensitive at 10 μM psychosine. More critically, they are an order of magnitude more sensitive than indicated by studies described in Zaka et al. (Mol. Cell Neurosci. 30 (3): 398-407 (2005)) on immortalized cell lines. The sensitivity of human CNS precursor cells was also examined and it was found that human fetal neuroepithelial stem cells (hNSCs) are at least as vulnerable to PC and GPC as are rat OPCs.

### Effects on Cytoskeleton

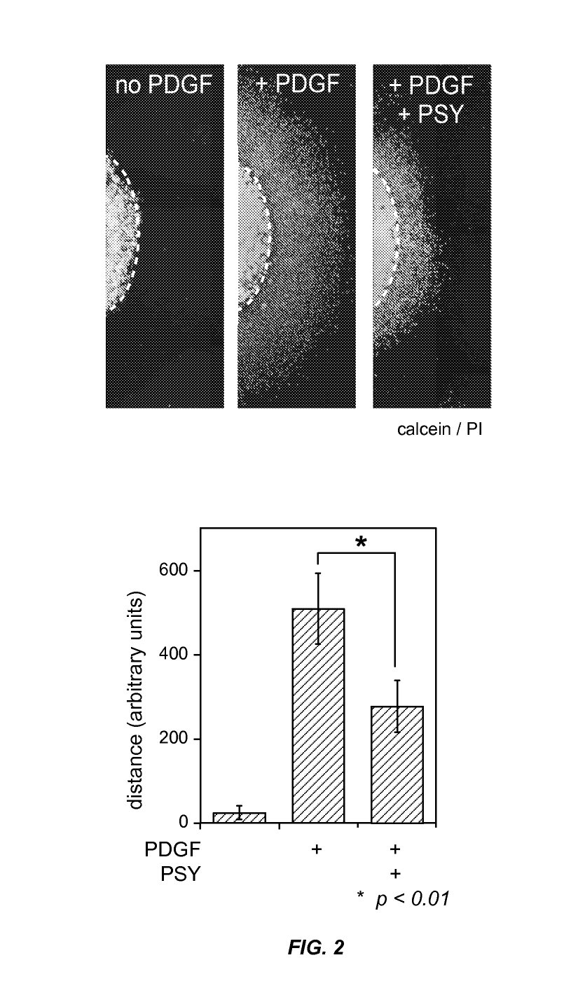

Another aspect of psychosine function is to cause alterations in the cytoskeleton, which in dividing cells is manifested as a failure of migration. This is shown in FIG. 2. The migration of oligodendrocyte progenitor cells outward from a drop of cells was examined. Cells were stained with calcein to label living cells and propidium iodide (PI) to label dead cells. The lack of PI labeled cells shows that this is not a lethal level of exposure. Exposure to even 1 μM psychosine is enough to greatly inhibit cell migration. Dominant negative c-Cb1 expression did not rescue the cells from these effects of psychosine. Studies also showed that psychosine causes changes in calcium regulation and lysosomal function. This effect was observed on multiple cell types.

### Analysis of FDA Approved Drugs

Experiments were conducted to identify FDA-approved agents that rescue precursor cells of the CNS from the effects of the lipids thought to be toxic in KD, GD and MLD. To identify therapeutic agents that could be candidates for relatively rapid translation to the clinic, candidate agents were screened (based on cellular and molecular analyses) and unbiased drug screens were conducted using the NINDSII library of 1040 compounds approved by the FDA or in clinical trials. The intent of these screens was to discover previously unknown activities of agents for which pharmacokinetic and toxicological information is already available. The initial screening assays used are for (i) protection against OPC death induced by lethal concentrations of toxic lipids (48 hr exposure) and (ii) protection of OPC division induced by exposure to sublethal concentrations of toxic lipids.

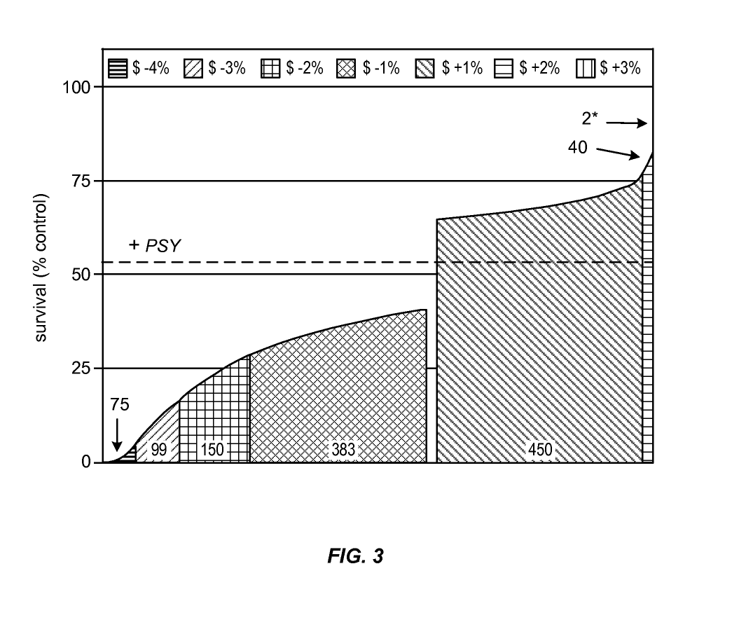

In this screen, a library of 1040 compounds, either approved for use or in clinical trials, were examined at three different doses for their ability to protect against psychosine-induced cell death. Forty compounds were found that caused a significant rescue of survival from the lethal effects of psychosine exposure (and two false positives, indicated by the *) (FIG. 3).

A similar analysis was conducted with the same 1040 agents, but with concentrations of psychosine that suppressed cell division, but did not cause appreciable cell death. Again, a large number of compounds that rescued cell division were found. Surprisingly, the overlap between compounds that rescued both cell survival and cell division was minimal. Chlorotrianisene, clofoctol, colforsin and tulobuterol were the compounds that rescued both cell survival and cell division.

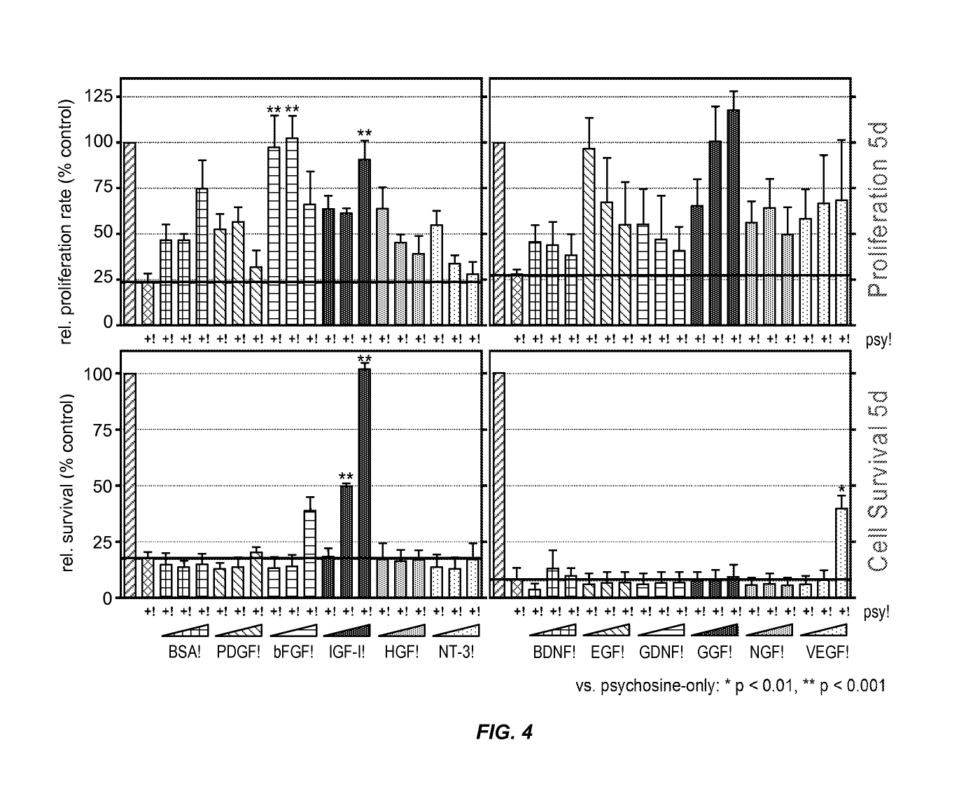

Similar screens of proteins were conducted. Multiple proteins were found that rescue cell division but fewer that impact survival. The compounds examined were platelet-derived growth factor, fibroblast growth factor-2, epidermal growth factor, hepatocyte growth factor, neurotrophin-3, glial-derived neurotrophic factor, glial growth factor (neuregulin), vascular endothelial growth factor (VEGF), insulin-like growth factor-I (IGF-1) and insulin-like growth factor-II (IGF-2). Only IGF-1 and VEGF had the ability to protect both cell division and cell survival (FIG. 4).

As set forth above, previous studies showed the potential ability of IGF-1 to protect against psychosine toxicity in an immortalized cell line characterized as an oligodendrocyte progenitor cell line (Zaka et al., 2005, Mol. Cell. Neurosci. 30:398-407). However, this cell line is highly resistant to psychosine and requires exposure to 50 μM psychosine in order to cause marked cell death over 24 hours. This is more than an order of magnitude greater level of resistance than seen with primary oligodendrocyte progenitor cells. Moreover, the levels of IGF-1 required to prevent cell death at a significant level are extremely high, with a slight reduction seen at exposure levels of 100 ng/ml and marked protection only achieved with doses of one μg per ml. Thus, based on Zaka et al., the levels of IGF-I required to obtain rescue of these cells are generally considered to be in the supra-physiological range and offer no prediction of the outcomes that can be obtained at physiologically relevant exposure levels. At best, Zaka et al. indicates that, supra-physiological levels of an agonist for a tyrosine kinase receptor can protect against psychosine-induced cell death in an immortalized cell line that is intrinsically resistant to psychosine, wherein the cells are resistant to psychosine at levels over 10 times greater than those that affect normal oligodendroctye progenitor cells.

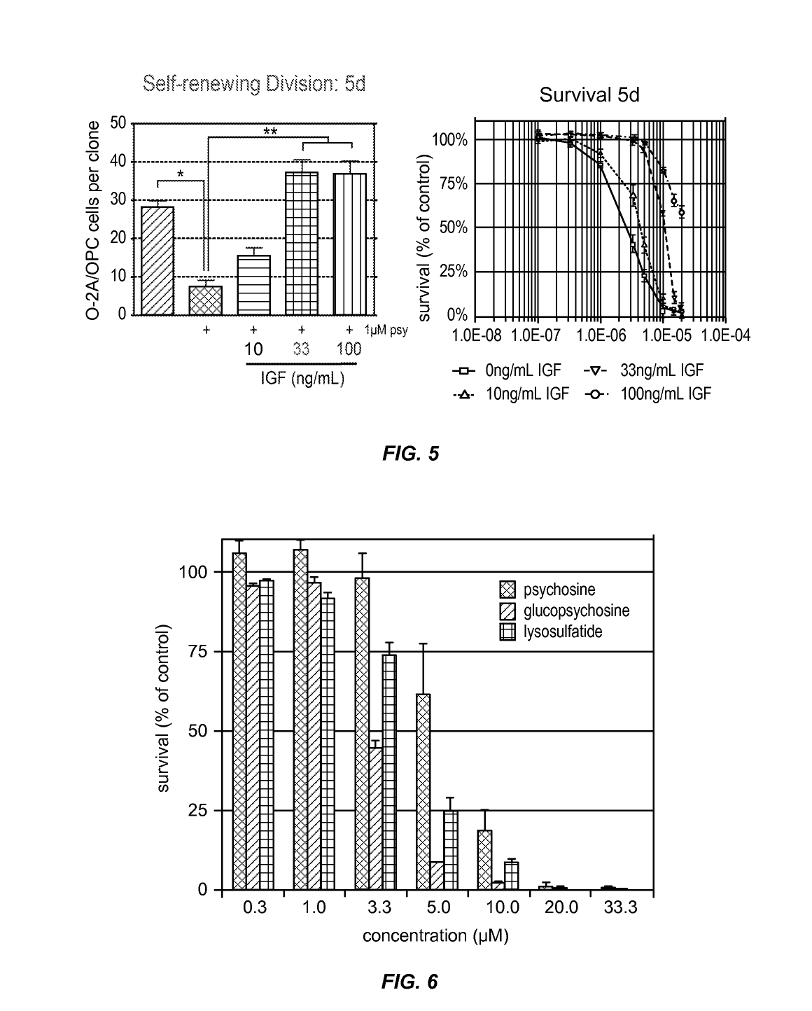

The effect of IGF-1 on rescuing division and survival of primary oligodendrocyte progenitor cells is shown in FIG. 5. These data show that beneficial effects on survival are seen at dosages of 10 ng/ml (in contrast with the marginal effect of 100 ng/ml IGF-I on the immortalized cell line studied by Zaka et al.). Rescue of cell division is also achieved at physiologically relevant dosages of IGF-1, something that has not been previously observed.

To better define the properties of agents that are most effective in rescuing cells from the toxic effects of psychosine, all of the agents that rescue both cell survival and cell division were examined in the context of other properties relevant to the disease manifestations of Krabbe disease. The effects of cytoskeletal collapse and changes in lysosomal pH were examined. It was found that agents that rescue both division and survival also prevent cytoskeletal collapse. Moreover, the pH of cells exposed to psychosine becomes more alkaline and is moved outside of the pH range found in normal cells not exposed to psychosine. The agents that protect both division and survival also restore lysosomal pH to a normal range. However, all of the agents that rescue cell division examined for their effects on lysosomal pH also restore normal levels of pH. Thus, rescuing lysosomal pH is not sufficient to rescue cell survival. Further, agents chosen solely on the basis of rescue of lysosomal pH are not sufficient to also function as rescue agents in cell survival.

Since lysosomal storage disorders are so rare, there is a strong disincentive to invest heavily in treatments. Therefore, there is a clear need to identify agents that would be of use in the treatment of multiple lysosomal storage disorders. As it is so difficult to find agents that rescue both division and survival, it was reasonable to assume that agents that are able to rescue against multiple toxicities do not exist. Nonetheless, due to the importance of such a goal, the agents that protect against both cell death and suppression of cell division caused by exposure to psychosine were examined in order to determine their potential utility in protection against the toxic effects of glucopsychosine (elevated in Gaucher's disease) and of lysosulfatide (elevated in metachromatic leukodystrophy). These diseases were chosen as they come from two functionally different disease groups as defined by a cellular assay for lysosomal function wherein BODIPY-LacCer uptake is detected.

It was found that glucopsychosine and lysosulfatide are toxic for primary oligodendrocyte progenitor cells and other primary cells of the central nervous system at exposure levels in the low micromolar range at which toxicity of psychosine is seen (FIG. 6).

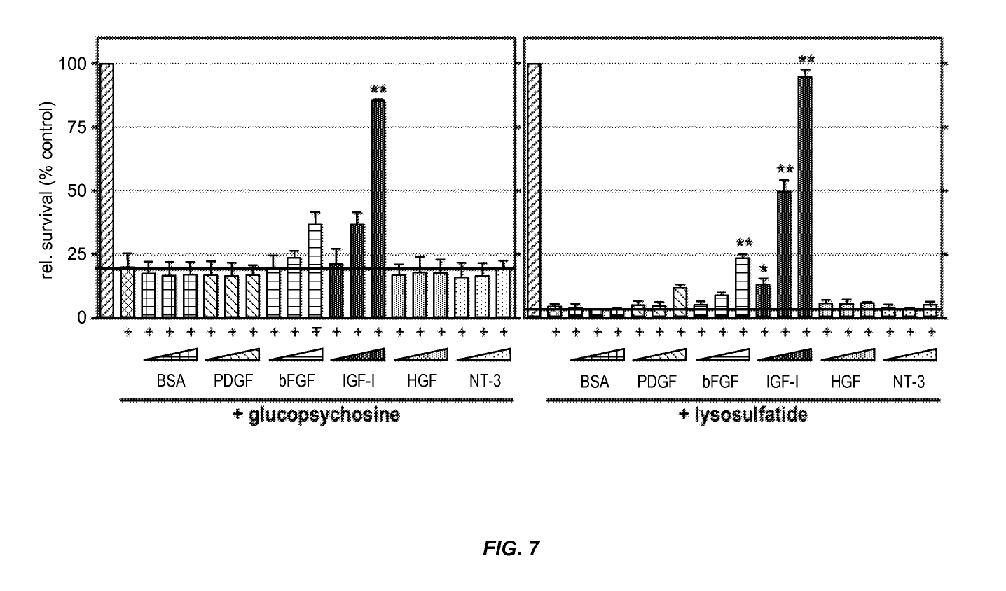

The ability of agents that protect against psychosine mediated suppression of cell division and cell survival to also protect against the toxicity of glucopsychosine and lysosulfatide was examined. When a group of bioactive proteins was examined, it was again found that this was a unique property of IGF-1 (FIG. 7). When the rescue competence of IGF-1 was examined at longer time points, IGF-1 was found to be similarly effective against lysosulfatide and glucopsychosine as it was against psychosine, with survival data shown at 5 days for lysosulfatide.

### Example 2

### In Vivo Studies

In order to determine whether agents identified in in vitro studies are capable of providing an in vivo benefit, the effects of IGF-1 were examined in twitcher −/− mice, an accepted model of Krabbe disease. The twitcher −/− mice harbor a mutation in galactocerebosidase that mimics Krabbe disease. The strategy for in vivo analyses was to determine whether FDA-approved compounds of potential interest improve neurological symptoms and/or extend survival times of twi−/− mice. These studies used techniques employed in studies on toxicity of chemotherapeutic agents for the CNS (See Dietrich et al. J. Biol. 5 (7): e22 (2006); Han et al. CNS J. Biol. 7: e12 (2008)).

In in vivo experiments, IGF-I was administered daily, beginning 10 days after birth, through i.p. injection (100 μg/kg, which is below dosages used in other studies using peripheral administration (See Aberg et al. J. Neurosci. 20: 2896-2903 (2000)). This time point is earlier than the expression of neurological symptoms but is considerably later than that used in attempts to treat these mice by HSC transplants, enzyme replacement therapy or both combined (See Qin et al. Mol. Genet. Metab. 107: 186-196 (2012)). Mice were evaluated at 24 days of age to enable analysis of cell division in vivo. Comparative analysis after 40 days was not possible due to death of the control (saline-treated) twi−/− mice shortly after 40 days of age.

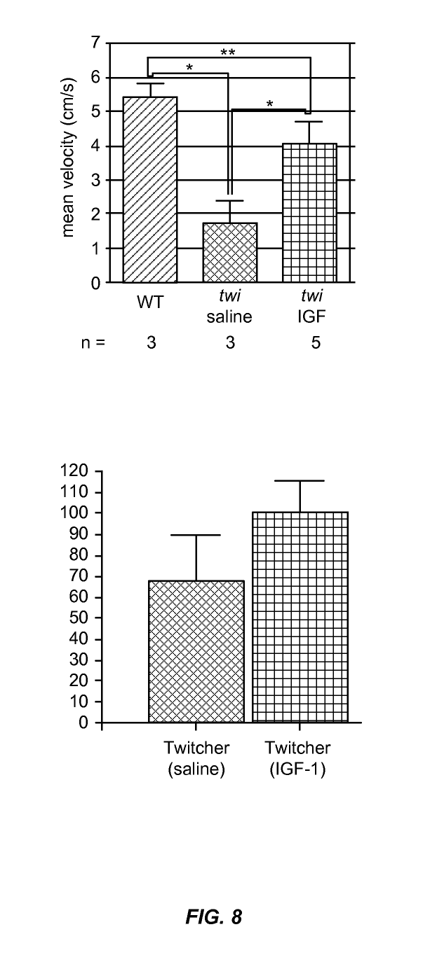

Experiments demonstrated that improvement is seen even when treatment is started as late as 10 days after birth (a much later time point than is used in studies on rescue by combinations of hematopoietic stem cell transplantation and gene therapy). Drugs are delivered once daily by i.p. administration (using 100 μg/kg, which provide benefit (see FIG. 8). All animals are marked and genotyped within the first week after birth to identify +/+, +/− and −/− pups. Outcome measures such as (i) weight gain, motor activity, rotorod analysis, auditory brainstem response and survival are analyzed.

Films of the behavior of IGF-I treated, 24-day-old twi−/− mice demonstrate that these mice are essentially normal in appearance and can barely be distinguished from wild-type counterparts. These data are shown in still images representing one picture per second over a ten-second period. In these still images, wild-type twi littermates are shown to move freely in their cages. For example, a wild-type littermate traverses the perimeter of the cage, showing normal body extension and climbing behavior. In control (saline alone) twi−/− mice, as is typical at this age, the mice are shown as hunched over, lacking normal body extension and little locomotor behavior. In addition, the saline control twi−/− animals showed disheveled fur and, when they move, they show signs of considerable weakness (with poor weight support) and constant shivering behavior. The saline control twi−/− mice also weigh less than wild-type littermates.

The images of the IGF-1 treated twi−/− mouse, in contrast, show normal body extension, sleek fur and evidence of extensive locomotor behavior. The treated animals also show normal weight support, an absence of shivering and normal weight gain. Analysis of movement velocity (cm/sec) shows that treated mice show statistically normal locomotor activity (FIG. 8).

Mice were sacrificed at Day 25 to examine OPC division in vivo (FIG. 8). Analysis for 3 twi−/− control mice and 3 IGF-1 treated mice showed ˜45% more dividing OPCs detected in the corpus callosum of IGF-1 treated mice than in saline treated mice.

In vivo studies revealed outcomes consistent with the in vitro studies. At age P40, there was a ˜50% reduction in the number of dividing OPCs in the untreated saline control twi−/− animals. Even at P15, however, before any neurological symptoms or signs of inflammation are apparent, there was a significant 18% reduction in dividing progenitor cells. Similar in vivo results were obtained by daily administration of colforsin.

1. A method of treating a lysosomal storage disease (LSD) in a subject comprising administering to the subject an effective amount of a first agent that inhibits cell death and reduces suppression of cell division of primary central nervous system precursor cells in the presence of one or more toxic substances, wherein the toxic substances are known to accumulate in cells affected by a lysosomal storage disease.

2. The method of claim 1, wherein the first agent is selected from the group consisting of IGF-1, VEGF, chlorotrianisene, clofoctal, and colforsin.

3. The method of claim 1, wherein the first agent is a small molecule.

4. The method of claim 3, wherein the small molecule is selected from the group consisting of chlorotrianisene, clofoctal, and colforsin.

5. The method of claim 1, wherein the LSD is a sphingolipid storage disorder.

6. The method of claim 5, wherein the sphingolipid storage disorder is selected from the group consisting of gloiboid-cell leukodystrophy (Krabbe Disease), metachromatic leukodystrophy, and Gaucher's Disease.

7. The method of claim 1, wherein the one or more toxic substances are selected from the group consisting of glucopsychosine, lysosulfatide, and psychosine.

8. The method of claim 1, wherein the first agent inhibits cell death and reduces suppression of cell division of primary central nervous system precursor cells in the presence of at least two toxic substances.

9. The method of claim 1, wherein the first agent is IGF-1 and the dosage is 0.01-0.40 mg/kg daily.

10. The method of claim 9, wherein the IGF-1 dosage is 0.01-0.40 mg/kg at least twice daily.

11. The method of claim 9, wherein the IGF-1 dosage is 0.01-0.12 mg/kg at least once daily.

12. The method of claim 11, wherein the IGF-1 dosage is 0.08-0.24 mg/kg daily.

13. The method of claim 1, wherein the first agent is VEGF and the LSD is not gloiboid-cell leukodystrophy (Krabbe Disease).

14. The method of any of claims 9 to 13 claim 9, wherein IGF or VEGF is administered by a pump.

15. The method of claim 1, further comprising administering to the subject a second agent that inhibits cell death and reduces suppression of cell division of primary central nervous system precursor cells in the presence of one or more toxic substances.

16. The method of claim 15, wherein the first and second agent are selected from the group consisting of IGF-1, VEGF, chlorotrianisene, clofoctal, and colforsin.

17. The method of claim 1, further comprising treating the subject with one or more treatments selected from the group consisting of a bone marrow transplant, enzyme replacement, and gene therapy.

18. The method of claim 1, wherein the first agent inhibits cell death and reduces suppression of cell division of primary oligodendrocyte precursor cells in the presence of the one or more toxic substances.

19. A method of treating a lysosomal storage disease in a subject comprising
a. Selecting one or more agents that inhibits cell death and reduces suppression of cell division of primary central nervous system precursor cells in the presence of one or more toxic substances, wherein the toxic substances are known to accumulate in cells affected by a lysosomal storage disease, and
b. Administering an effective amount of the one or more agents to the subject.

20. The method of claim 19, wherein the one or more agents is selected from the group consisting of IGF-1, VEGF, chlorotrianisene, clofoctal, and colforsin.

21. The method of claim 19, wherein the LSD is a sphingolipid storage disorder.

22. The method of claim 21, wherein the sphingolipid storage disorder is selected from the group consisting of gloiboid-cell leukodystrophy (Krabbe Disease), metachromatic leukodystrophy, and Gaucher's Disease.

23. The method of claim 19, wherein the one or more toxic substances are selected from the group consisting of glucopsychosine, lysosulfatide, and psychosine.

24. The method of claim 19, wherein the one or more agents inhibit cell death and reduce suppression of cell division of primary central nervous system precursor cells in the presence of at least two toxic substances.

25. The method of claim 19, wherein the one or more agents is IGF-1 and the dosage is 0.01-0.40 mg/kg daily.

26. The method of claim 25, wherein the IGF-1 dosage is 0.01-0.40 mg/kg at least twice daily.

27. The method of claim 25, wherein the IGF-1 dosage is 0.01-0.12 mg/kg at least once daily.

28. The method of claim 27, wherein the IGF-1 dosage is 0.08-0.24 mg/kg daily.

29. The method of claim 19, wherein the one or more agents comprises VEGF and the LSD is not globoid-cell leukodystrophy (Krabbe Disease).

30. The method of claim 25, wherein IGF or VEGF is administered by a pump.

31. The method of claim 19, further comprising treating the subject with one or more treatments selected from the group consisting of a bone marrow transplant, enzyme replacement, and gene therapy.

32. The method of claim 19, wherein the primary central nervous system precursor cells are glial precursor cells.

33. A method of screening for an agent for treating lysosomal storage disease (LSD) comprising:
a. Detecting a level of cell survival of a primary central nervous system cell in the presence of one or more toxic substances, wherein the one or more toxic substances are known to accumulate in cells affected by a lysosomal storage disease;
b. Detecting a level of cell division of a primary central nervous system cell in the presence of the one or more toxic substances, an agent that increases the level of cell survival and increases the level of cell division of the primary central nervous system cell in the presence of the one or more toxic substances as compared to the level of cell survival and cell division in the absence of the agent indicates an agent that treats lysosomal storage disease.

34. The method of claim 33, wherein the steps of detecting survival and division of the primary central nervous system cell are performed in vitro.

35. The method of claim 33, wherein the primary central nervous system cell is a precursor cell.

36. The method of claim 35, wherein the precursor cell is a glial precursor cell.

37. The method of claim 36, wherein the glial precursor cell is capable of giving rise to an oligodendrocyte.

38. The method of claim 35, wherein the precursor cell is an oligodendroctye-type-2 astrocyte (O-2A) precursor cell.

39. The method of claim 35, wherein the one or more toxic substances comprise a lipid.
